(19) United States
(12) Patent Application Publication
(10) Pub. No.: US 2026/0083432 A1
(43) Pub. Date: Mar. 26, 2026

(54) DETECTION OF ROTATIONAL IMAGING CATHETER TWIST USING IMAGE CORRELATION

(71) Applicant: Boston Scientific Scimed, Inc., Maple Grove, MN (US)

(72) Inventors: Timothy G. Curran, St. Paul, MN (US); Wenuang Li, Los Gatos, CA (US); Corydon Carlson, Stillwater, MN (US)

(73) Assignee: Boston Scientific Scimed, Inc., Maple Grove, MN (US)

(21) Appl. No.: 19/338,520

(22) Filed: Sep. 24, 2025

Related U.S. Application Data
(60) Provisional application No. 63/699,279, filed on Sep. 26, 2024.

(51) Int. Cl. A61B 8/00; A61B 8/08; A61B 8/12; G06T 7/73

(52) U.S. Cl. CPC A61B 8/4245; A61B 8/12; A61B 8/461; A61B 8/54; G06T 7/74; A61B 8/0891; G06T 2207/30244

(57) ABSTRACT

The present disclosure is directed towards detecting the potential for and/or buildup of torsional energy in a rotational imaging device. The disclosure can provide an alert to the user, to allow the user to recover from the situation before catastrophic complications (e.g., knotting of the core, or the like) occur. The disclosure provides to correlate sequential images captured by the rotational imaging device to detect the potential for and/or buildup of torsional energy in the device. Rotation between sequential images can be correlated to identify when torsional energy is building in the device.

### CROSS-REFERENCE TO RELATED APPLICATIONS

This patent application claims the benefit of priority under 35 U.S.C. § 119 to U.S. Provisional Patent Application No. 63/699,279, filed Sep. 26, 2024, which is herein incorporated by reference in its entirety.

### TECHNICAL FIELD

The present disclosure generally relates to rotating imaging devices and systems and can be implemented to detect the potential for twisting, or the buildup of torque, in the rotating core of the imaging device.

### BACKGROUND

Miniature imaging probes attached to a distal end of a catheter can be inserted into a patient to capture intracorporeal images. Often, such images are used to visualize internal anatomical structures of the patient. For example, an imaging probe (e.g., an ultrasound probe, an optical coherence tomography (OCT) probe, etc.) can be used to visualize vasculature structure, visualize pulmonary structure, or the like. Often, the imaging probe is attached to a distal end of a catheter which is inserted into the patient (e.g., into the patient's cardiac arteries, into the patient's pulmonary lumens, etc.). The imaging probe includes an imaging device (e.g., ultrasound transducer, optical transducer, etc.) coupled to a core that extends between the distal end of the catheter and the proximal end of the catheter. At the proximal end of the catheter the core is coupled to equipment, such as, an imaging console. The equipment is configured to receive signals from the imaging device and render images of structure being visualized.

The equipment is often configured to rotate the core, which in turn rotates the imaging device at the distal end of the catheter. As such, cross-sectional views of the patient's anatomy can be captured. For example, in the case of intravascular imaging, the cross-sectional views can be used to visualize the structure of a patient's vasculature from inside the target vessel or artery, out through the surrounding blood column. This facilitates visualizing the luminal wall of the vessel or artery and any other structure proximal to the luminal wall, such as, for example, plaque.

It is well known that progressive accumulation of plaque within a patient's vasculature can lead to heart attack, stenosis (e.g., narrowing), or other disease states. Such rotational imaging technologies can be employed to determine both the plaque volume and the degree of stenosis. Further, such rotational imaging technologies can be employed to assess the effects of interventions (e.g., stenting, balloon dilation, etc.).

To visualize a representative portion of the patient's anatomy, the imaging device is often rotated while the catheter is being moved through the lumen (e.g., pulled proximally or pushed distally). In some cases, where the imaging device is rotated while the catheter is distally advanced through tortuous or restricted anatomy, the imaging device may bind causing the core to “wind up” with torsional energy. This torsional energy can cause complications to the procedure, such as, twisting and/or kinking of the core. Further, such torsional energy can introduce distortions into the images.

### BRIEF SUMMARY

The present disclosure provides methods and computing systems configured to correlate sequential images captured by the rotating imaging device to detect the potential for and/or buildup of torsional energy. Methods and computing systems described herein also provide an alert to the user to enable the user to recover from the situation before catastrophic complications (e.g., knotting of the core, or the like) occur.

In some embodiments, the disclosure can be implemented as a computer-implemented method, such as might be implemented by a rotational imaging device controller, intravascular ultrasound (IVUS) imaging system, or the like. The method can comprise receiving a series of image frames captured by a rotational imaging device, where the series of image frames comprises at least a first image frame and a second image frame successive to the first image frame; identifying an angle of rotation between the first image frame and the second image frame; and generating, responsive to the determined angle of rotation, a control signal comprising an indication of a potential twisting of the rotational imaging device.

With some embodiments, the method can comprise cross-correlating the first image frame and the second image frame to identify the angle of rotation.

With some embodiments, the method can comprise filtering the series of image frames to generate a filtered series of image frames, wherein identifying the angle of rotation between the first image frame and the second image frame comprising identifying the angle of rotation from the filtered series of image frames.

With some embodiments of the method, the control signal comprises an indication of a graphical alert to be displayed on a display.

With some embodiments, the method can comprise determining whether the determined angle of rotation is greater than or equal to a rotation threshold; and generating the control signal based on a determination that the determined angle of rotation is greater than or equal to the rotation threshold.

With some embodiments of the method, the angle of rotation is a first angle of rotation, and the series of image frames comprises at least a third image frame successive to the second image frame, and the method can further comprise identifying a second angle of rotation between the second image frame and the third image frame; and generating the control signal responsive to the first and the second angles of rotation.

With some embodiments of the method, the rotational imaging device is an intravascular ultrasound (IVUS) catheter comprising a distal imaging core coupled to a proximal motor drive unit connector via a driveshaft and wherein the potential twisting of the rotational imaging device corresponds to a potential winding up of the driveshaft.

In some embodiments, the disclosure can be implemented as a rotational imaging device control system, an intravascular ultrasound (IVUS) imaging system, or the like where the system is configured to be coupled to a motor drive unit and an imaging catheter, such as an IVUS catheter. In such embodiments, the system can comprise processing circuitry and a memory comprising instructions, which when executed cause the system to receive, from the IVUS catheter, a series of image frames captured by the IVUS catheter, where the series of image frames comprises at least a first image frame and a second image frame successive to the first image frame; identify an angle of rotation between the first image frame and the second image frame; generate, responsive to the determined angle of rotation, a graphical indication of a potential twisting of the IVUS catheter; and display on a display coupled to the IVUS imaging system the graphical indication.

With some embodiments of the system, the instructions when executed by the processing circuitry further cause the system to cross-correlate the first image frame and the second image frame to identify the angle of rotation.

With some embodiments of the system, the instructions when executed by the processing circuitry further cause the system to filter the series of image frames to generate a filtered series of image frames, wherein the angle of rotation between the first image frame and the second image frame is identified from the filtered series of image frames.

With some embodiments of the system, the instructions when executed by the processing circuitry further cause the system to determine whether the determined angle of rotation is greater than or equal to a rotation threshold; and generate the control signal based on a determination that the determined angle of rotation is greater than or equal to the rotation threshold.

With some embodiments of the system, the angle of rotation is a first angle of rotation, the series of image frames comprises at least a third image frame successive to the second image frame, and the instructions when executed by the processing circuitry further cause the system to identify a second angle of rotation between the second image frame and the third image frame; and generate the control signal responsive to the first and the second angles of rotation.

With some embodiments of the system, the imaging catheter comprises a distal imaging core coupled to a proximal motor drive unit connector via a driveshaft and wherein the potential twisting of the imaging catheter corresponds to a potential winding up of the driveshaft.

With some embodiments of the system, the system comprises the motor drive unit and the imaging catheter.

With some embodiments of the system, the imaging catheter is an IVUS catheter, and the system comprises the IVUS catheter.

In some embodiments, the disclosure can be implemented by a non-transitory computer-readable storage device. The storage device can comprise instructions that when executed by a processor of a rotational imaging device control system, such as, a processor of an intravascular ultrasound (IVUS) imaging system, cause the system to receive, from an IVUS catheter coupled to a motor drive unit, a series of image frames captured by the IVUS catheter, where the series of image frames comprises at least a first image frame and a second image frame successive to the first image frame; identify an angle of rotation between the first image frame and the second image frame; generate, responsive to the determined angle of rotation, a graphical indication of a potential twisting of the IVUS catheter; and display on a display coupled to the IVUS imaging system the graphical indication.

With some embodiments of the storage device, the instructions when executed by the processor further cause the system to cross-correlate the first image frame and the second image frame to identify the angle of rotation.

With some embodiments of the storage device, the instructions when executed by the processor further cause the system to filter the series of image frames to generate a filtered series of image frames, wherein the angle of rotation between the first image frame and the second image frame is identified from the filtered series of image frames.

With some embodiments of the storage device, the instructions when executed by the processing circuitry further cause the system to determine whether the determined angle of rotation is greater than or equal to a rotation threshold; and generate the control signal based on a determination that the determined angle of rotation is greater than or equal to the rotation threshold.

With some embodiments of the storage device, the imaging catheter comprises a distal imaging core coupled to a proximal motor drive unit connector via a driveshaft and wherein the potential twisting of the IVUS catheter corresponds to a potential winding up of the driveshaft.

With some embodiments of the storage device, the imaging catheter is an IVUS catheter.

### BRIEF DESCRIPTION OF THE DRAWINGS

To easily identify the discussion of any element or act, the most significant digit or digits in a reference number refer to the figure number in which that element is first introduced.

FIG. 1 illustrates an embodiment of a rotational imaging system in the form of an intravascular ultrasound (IVUS) imaging system.

FIG. 2A and FIG. 2B illustrate an embodiment of a rotational imaging device, in the form an IVUS catheter, that can be used with the rotational imaging systems of the present disclosure.

FIG. 3 illustrates an embodiment of a computer subsystem that can be used with the rotational imaging systems of the present disclosure to detect the buildup of torsional energy in a connected rotational imaging device.

FIG. 4 illustrates an embodiment of a logic flow to detect the buildup of torsional energy in a rotational imaging device.

FIG. 5A, FIG. 5B, and FIG. 5C illustrate an example series of rotational imaging frames.

FIG. 6 illustrates another embodiment of a logic flow to detect the buildup of torsional energy in a rotational imaging device.

FIG. 7 illustrates another embodiment of a computer subsystem that can be used with the rotational imaging systems of the present disclosure to detect the buildup of torsional energy in a connected rotational imaging device.

FIG. 8 illustrates another embodiment of a logic flow to detect the buildup of torsional energy in a rotational imaging device.

FIG. 9 illustrates an embodiment of a computer-readable storage medium.

FIG. 10 illustrates an embodiment of a computing system configured to implement any of the methods, techniques, or logic flows detailed herein.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

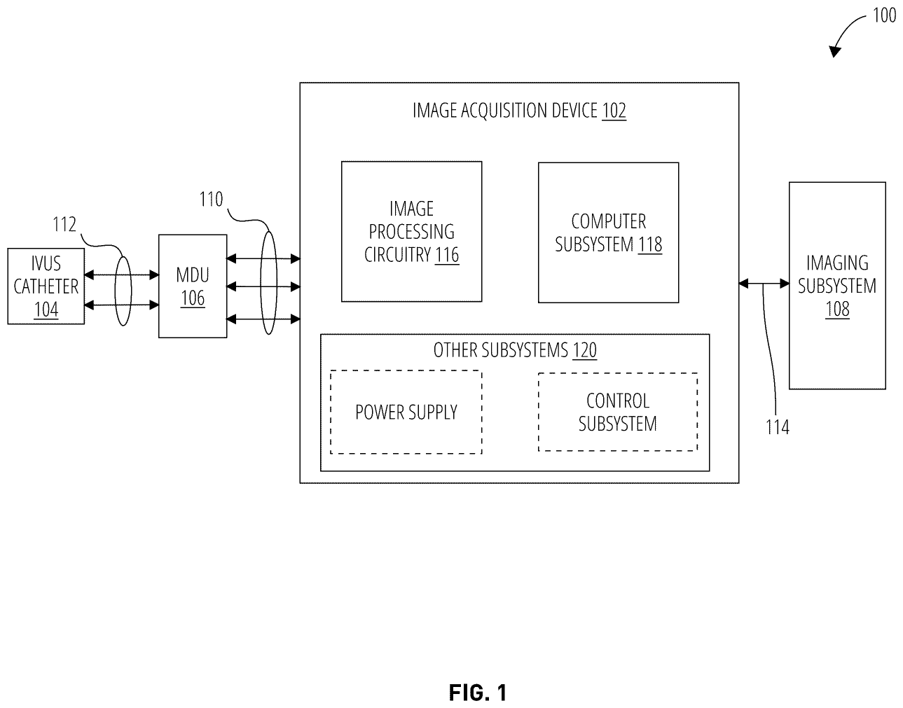

As introduced above, the disclosure provides methods and computer systems to detect potential for and/or actual twisting of a rotational imaging core driveshaft. As used herein, the term “twisting” means a difference in the rate of rotation between the proximal end of the driveshaft and the distal end of the drive shaft. As such, an example rotational imaging system is described. Although the disclosure can be implemented to detect the build-up of torsional energy in any rotational imaging system, an intravascular ultrasound (IVUS) system is used in the balance of the disclosure for purposes of clarity of presentation only and not as a necessary limitation. FIG. 1 illustrates an example IVUS imaging system 100. The IVUS imaging system 100 includes an image acquisition device 102, an IVUS catheter 104, a motor drive unit (MDU) 106, and an imaging subsystem 108. The image acquisition device 102 is coupled to the IVUS catheter 104 via the MDU 106 and is also coupled to the imaging subsystem 108. In particular, the image acquisition device 102 is coupled to the MDU 106 via the MDU bus 110 while the MDU 106 is coupled to the IVUS catheter 104 via the catheter bus 112. In some embodiments, the MDU bus 110 and the catheter bus 112 can be transmission lines (or other conductors) arranged to convey signals between the various components. For example, the MDU bus 110 and catheter bus 112 can be arranged to transmit radio frequency signals (e.g., control signals, ultrasound pulse generation signals, ultrasound signals, or the like) between the indicated components of the IVUS imaging system 100.

In general, the image acquisition device 102 is configured to control the MDU 106 and receive signals from the IVUS catheter 104, via the MDU 106. Further, the image acquisition device 102 is configured to process the received signals to generate images and convey the images to the imaging subsystem 108. To that end, the image acquisition device 102 is coupled to the imaging subsystem 108 via the imaging subsystem bus 114, which can be a wired connection or a wireless connection. As a specific example, imaging subsystem bus 114 can be an Ethernet connection. In some examples, the imaging subsystem 108 can be a display, a tablet computer, or other device configured to display images rendered by image acquisition device 102. It is noted that although the imaging subsystem 108 is depicted external to image acquisition device 102, with some embodiments, imaging subsystem 108 can be incorporated into the same housing as image acquisition device 102. Further, in some embodiments, the IVUS catheter 104, the MDU 106, the imaging subsystem 108, and/or the image acquisition device 102 can be combined into a single device.

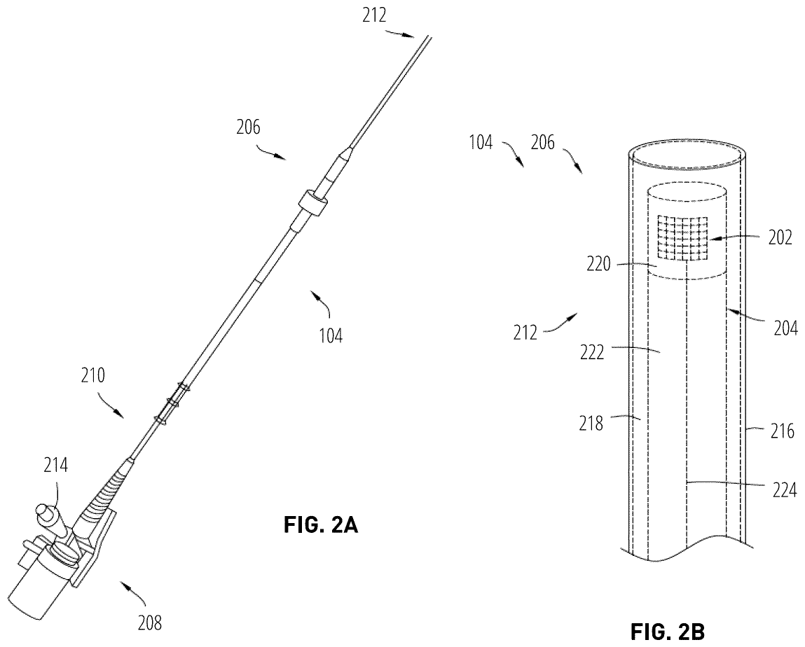

The image acquisition device 102 includes an image processing circuitry 116, computer subsystem 118, and other subsystems 120 (e.g., power supply circuitry, control circuitry, etc.) In general, the present disclosure provides an improvement to computing technology and/or IVUS imaging equipment in that the image acquisition device 102 can be configured to detect the potential for and/or actual twisting of the driveshaft of the core (see FIG. 2B) of the IVUS catheter 104. The image processing circuitry 116 and/or computer subsystem 118 can be configured to correlate consecutive images captured by the IVUS catheter 104 to identify image rotation between consecutive images and to detect intra-procedure (or in real-time) the potential for and/or possibility of twisting of the core, which could lead to catastrophic complications. Prior to describing detailed examples of these embodiments, a general description of the components of the IVUS imaging system 100 and particularly the IVUS catheter 104 is provided.

FIG. 2A illustrates a side perspective view of the IVUS catheter 104 of the IVUS imaging system 100 of FIG. 1 and FIG. 2B is a side perspective view of a distal end 212 of an elongated member 206 of the IVUS catheter 104. In some embodiments, the other subsystems 120 are configured to power MDU 106 and send signal to IVUS catheter 104 and particularly one or more transducers 202 disposed in the IVUS catheter 104 to cause the IVUS catheter 104 to emit ultrasound signals.

Further, mechanical energy from MDU 106 may be used to drive a core 204 (or imaging core 204) disposed in the IVUS catheter 104. The one or more transducers 202 are further configured to receive acoustic signals (e.g., echo signals, or the like) and transmit these signals to image acquisition device 102 via catheter bus 112, the MDU 106, and MDU bus 110. In general, the received acoustic signals can be reflections (e.g., from structure within the patient's anatomy, or the like) of the ultrasound signals emitted by the transducer 202. These signals can be conveyed (e.g., via catheter bus 112 and MDU bus 110 to the image acquisition device 102 for processing by image processing circuitry 116 and/or computer subsystem 118.

In some embodiments, other subsystems 120 can be configured to control at least one of the frequency or duration of the electrical pulses transmitted from image acquisition device 102 to MDU 106 to control, for example, the speed of rotation of the imaging core 204, the acceleration of the rotation of the imaging core 204, the velocity or length of the pullback of the imaging core 204 by the MDU 106, or the like.

The IVUS catheter 104 includes an elongated member 206 and a hub 208. The elongated member 206 includes a proximal end 210 and a distal end 212. The proximal end 210 of the elongated member 206 can be coupled to the hub 208 and the distal end 212 of the elongated member 206 is configured and arranged for percutaneous insertion into a patient. Optionally, the IVUS catheter 104 may define at least one flush port, such as flush port 214. The flush port 214 may be defined in the hub 208. The hub 208 may be configured and arranged to couple to the MDU 106 of IVUS imaging system 100.

The elongated member 206 includes a sheath 216 with a longitudinal axis 224 (e.g., a central longitudinal axis extending axially through the center of the sheath 216 and a lumen 218 disposed in the sheath 216. The sheath 216 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

An imaging core 204 is disposed in the lumen 218. The imaging core 204 includes an imaging device 220 coupled to a distal end of a driveshaft 222. The imaging device 220 includes any number of transducers 202. In some embodiments, for example as shown in these figures, an array of transducers 202 can be mounted to the imaging device 220. For example, there can be two, three, four, five, six, seven, eight, nine, ten, twelve, fifteen, sixteen, twenty, twenty-five, fifty, one hundred, five hundred, one thousand, or more transducers. Alternatively, a single transducer may be employed. When multiple transducers 202 are employed, the transducers 202 can be configured into any suitable arrangement including, for example, an annular arrangement, a rectangular arrangement, or the like. Further, where multiple transducers 202 are employed, they can be disposed at various angles with respect to the longitudinal axis 224 and/or angle of rotation about the longitudinal axis 224 of the elongated member 206.

The one or more transducers 202 may be formed from materials capable of transforming applied electrical pulses to pressure distortions on the surface of the one or more transducers 202, and vice versa. Examples of suitable materials include piezoelectric ceramic materials, piezocomposite materials, piezoelectric plastics, barium titanates, lead zirconate titanates, lead metaniobates, polyvinylidene fluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like).

As outlined above, the driveshaft 222 is rotatable. For example, the driveshaft 222 can be rotated manually. In other embodiments, the driveshaft 222 can be rotated using a computer-controlled drive mechanism (e.g., MDU 106). Rotation of the driveshaft 222 causes the imaging device 220 and the transducers 202 attached to the imaging device to be rotated. Signals emitted and received by the transducers 202 can be used to form radial cross-sectional image of the anatomy (e.g., vasculature, etc.) as described above. However, where the distal end 212 of the IVUS catheter 104 is advanced through tortious and/or narrow anatomy while the imaging device 220 is rotated, friction between the imaging device 220 and the sheath 216 may cause torsional energy to accumulate in the driveshaft 222. Said differently, in such scenarios, the distal end of the driveshaft 222 may rotate at a different rate than the proximal end of the driveshaft 222, causing the driveshaft to “twist” and/or buildup torsional energy.

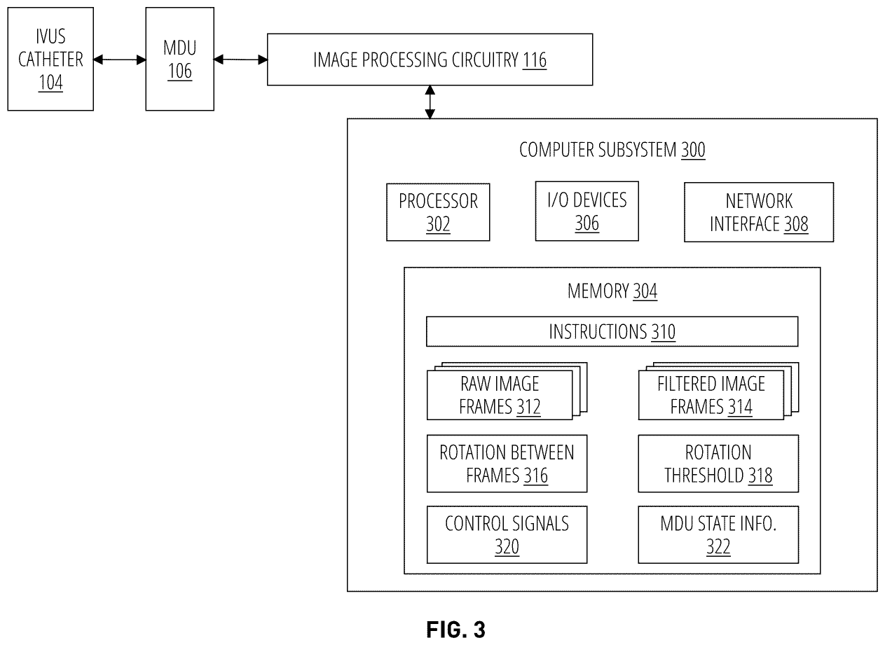

FIG. 3 illustrates an example of a computer subsystem 300, which can be implemented as part of the IVUS imaging system 100 of FIG. 1. For example, computer subsystem 300 could be implemented as computer subsystem 118 of IVUS imaging system 100 and configured to correlate consecutive images captured by IVUS catheter 104 and detect the potential for and/or actual twist of the driveshaft 222. Image processing circuitry 116 can include analog processing circuitry configured to transform electrical signals received from the IVUS catheter 104, and particularly from the transducer 202, into digital signals that can be processed by computer subsystem 300.

Computer subsystem 300 can be any of a variety of computing devices but will in general include processing circuitry and memory. In some embodiments, computer subsystem 300 can be incorporated into and/or implemented by a console of IVUS imaging system 100 as depicted in FIG. 1. With some embodiments, computer subsystem 300 can be a workstation or server communicatively coupled to MDU 106 and IVUS catheter 104. With still other embodiments, computer subsystem 300 can be provided by a cloud based computing device, such as, by a computing as a service (CaaS) system accessibly over a network (e.g., the Internet, an intranet, a wide area network, or the like). In some embodiments, computer subsystem 300 can be incorporated into image processing circuitry 116. That is, the processing circuitry of image processing circuitry 116 that is configured to transform the analog signals received from IVUS catheter 104 can also include processing circuitry configured to detect twisting as outlined herein. As a specific example, a combination image processing circuitry 116 and computer subsystem 300 could be implemented with a field programmable gate array (FPGA). However, for purposes of clarity of presentation only, computer subsystem 300 is depicted and described herein distinct from image processing circuitry 116.

Computer subsystem 300 can include processor 302, memory 304, input and/or output (I/O) devices 306, and network interface 308. The processor 302 may include circuity or processor logic, such as, for example, any of a variety of commercial processors. In some examples, processor 302 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 302 may include graphics processing portions and may include dedicated memory, multiple-threaded processing and/or some other parallel processing capability. In some examples, the processor 302 may be an application specific integrated circuit (ASIC) or a field programmable integrated circuit (FPGA).

The memory 304 may include logic, a portion of which includes arrays of integrated circuits, forming non-volatile memory to persistently store data or a combination of non-volatile memory and volatile memory. It is to be appreciated, that the memory 304 may be based on any of a variety of technologies. In particular, the arrays of integrated circuits included in memory 304 may be arranged to form one or more types of memory, such as, for example, dynamic random access memory (DRAM), NAND memory, NOR memory, or the like.

I/O devices 306 can be any of a variety of devices to receive input and/or provide output. For example, I/O devices 306 can include, a keyboard, a mouse, a joystick, a foot pedal, a display, a touch enabled display, a haptic feedback device, an LED, or the like.

Network interface 308 can include logic and/or features to support a communication interface. For example, network interface 308 may include one or more interfaces that operate according to various communication protocols or standards to communicate over direct or network communication links. Direct communications may occur via use of communication protocols or standards described in one or more industry standards (including progenies and variants). For example, network interface 308 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 308 can include logic and/or features to enable communication over a variety of wired or wireless network standards (e.g., 802.11 communication standards). For example, network interface 308 may be arranged to support wired communication protocols or standards, such as, Ethernet, or the like. As another example, network interface 308 may be arranged to support wireless communication protocols or standards, such as, for example, Wi-Fi, Bluetooth, ZigBee, LTE, 5G, or the like.

Memory 304 can include instructions 310, raw image frames 312, filtered image frames 314, rotation between frames 316, rotation threshold 318, control signals 320, and/or MDU state information 322. During operation, processor 302 can execute instructions 310 to cause computer subsystem 300 to receive raw image frames 312 from image processing circuitry 116. Processor 302 can further execute instructions 310 to filter and/or pre-process raw image frames 312 to generate filtered image frames 314. With some examples, processor 302 can execute instructions 310 to apply various image filtering algorithms to the raw image frames 312 (e.g., a Gaussian filter, a two-dimension (2D) matrix filter, a blur filter, a segmentation filter, or the like).

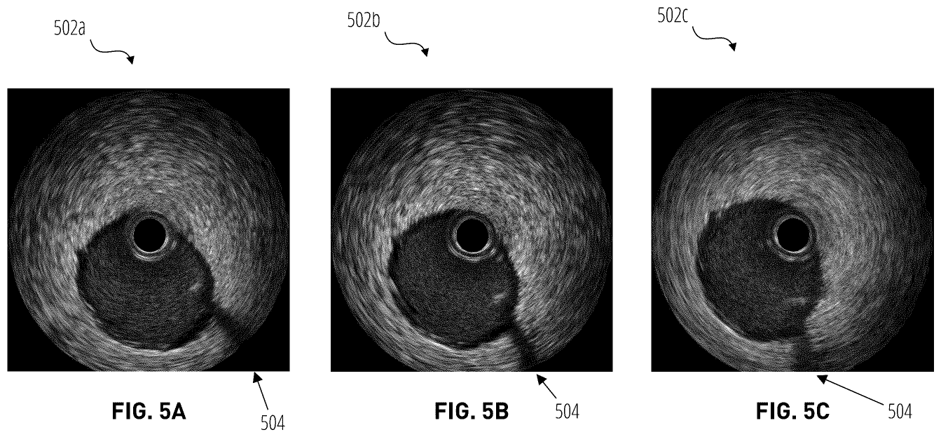

Processor 302 can execute instructions 310 to identify a rotation between successive frames of the raw image frames 312 and/or filtered image frames 314. For example, where processor 302 executes instructions 310 to generate filtered image frames 314 from raw image frames 312; processor 302 can execute instructions 310 to identify rotation between frames 316 from successive frames of filtered image frames 314. However, in other embodiments, processor 302 can execute instructions 310 to identify rotation between frames 316 from successive frames of raw image frames 312. This is described in greater detail below, for example, with reference to FIG. 5A, FIG. 5B, and FIG. 5C. However, in general processor 302 can execute instructions 310 to correlate an artifact or constant in the image frames and identify an angle of rotation of the artifact between successive frames. For example, an IVUS catheter (e.g., IVUS catheter 104, or the like) is often inserted over a guidewire. The guidewire will produce a shadow (or artifact) in the captured images. Processor 302 can execute instructions 310 to determine an angle or rotation between the guidewire shadow using a cross-correlation image processing algorithm.

Processor 302 can execute instructions 310 to determine whether the identified rotation between frames 316 exceeds a rotation threshold 318. Processor 302 can execute instructions 310 to generate control signals 320 responsive to a determination that rotation between frames 316 exceeds rotation threshold 318. In some examples, control signals 320 can be control signals to be sent to the MDU 106 to cause the MDU 106 to change (e.g., reduce or modulate the speed of rotation. In other examples, control signals 320 can be control signals to be sent to the MDU 106 to cause the MDU 106 to stop rotation. With still other examples, control signals 320 can be control signals to be sent to a display (e.g., a display of imaging subsystem bus 114, or the like) to cause the display to provide a graphical alert to the user of the potential for twisting of the driveshaft 222. With yet other examples, control signals 320 can be control signals to be sent to a speaker (e.g., a speaker of IVUS imaging system 100, or the like) to cause the speaker to provide an audible alert to the user of the potential for twisting of the driveshaft 222.

With some examples, processor 302 can execute instructions 310 to determine rotation between frames 316 based on filtered image frames 314 and MDU state information 322. For example, MDU state information 322 can include information such as the speed of rotation, an acceleration of rotation, current supplied to the motor in the MDU 106, or the like. Accordingly, the determined angle of rotation can be complemented with MDU state information 322. As a specific example, multiple rotation thresholds 318 may be provided based on the speed and/or acceleration of the MDU 106. As a specific example, a smaller rotation threshold 318 may be provided where the acceleration is positive while a larger rotation threshold 318 may be provided where the acceleration is negative.

It is to be appreciated that where acceleration of the MDU 106 is positive, the speed and potentially the torque applied to the driveshaft 222 will be increasing. Conversely, where acceleration of the MDU 106 is negative, the speed and possibly the torque applied to the driveshaft 222 will be decreasing. As such, a first value for rotation threshold 318 may be specified for instances where the acceleration of the MDU 106 is positive and a second value for rotation threshold 318 may be specified for instances where the acceleration of the MDU 106 is negative, where the second value is larger than the first value. As such, the smaller value for rotation threshold 318 will tighten the range within which rotation between the proximal and the distal ends of the driveshaft 222 is acceptable for instances where the acceleration of the MDU 106 is positive. Likewise, the larger value for rotation threshold 318 will widen the range within which rotation between the proximal and the distal ends of the driveshaft 222 is acceptable for instances where the acceleration of the MDU 106 is negative.

In some embodiments, different values for the rotation threshold 318 can be provided for different speeds of the MDU 106. For example, at higher speeds, a smaller value for rotation threshold 318 may be applied than for lower speeds.

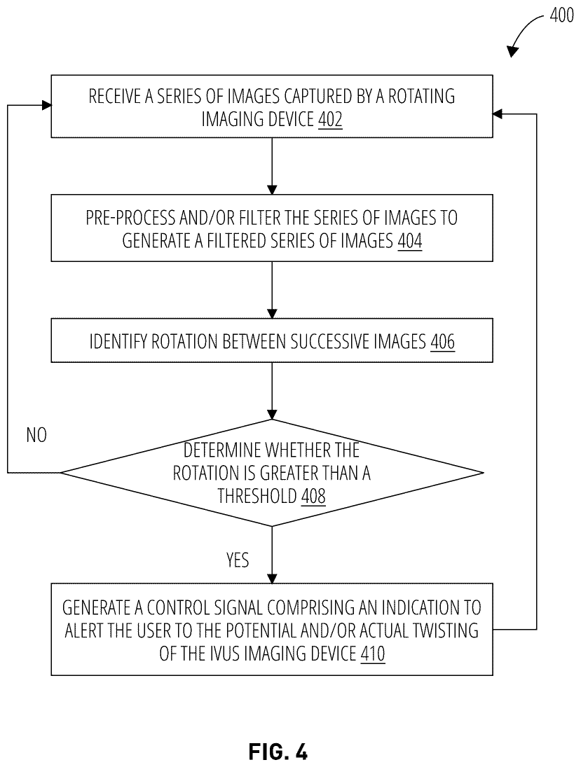

FIG. 4 illustrates a logic flow 400 to identify potential for and/or actual twisting of a rotational imaging driveshaft, according to some embodiments of the present disclosure. The logic flow 400 can be implemented by computer subsystem 300, which itself can be implemented by IVUS imaging system 100. Further, logic flow 400 will be described with reference to computer subsystem 300 for clarity of presentation. However, it is noted that logic flow 400 could also be implemented by an IVUS imaging system different than IVUS imaging system 100.

Logic flow 400 can begin at block 402. At block 402 “receive a series of images captured by a rotating imaging device” a series of images captured by a rotating imaging device can be received. For example, computer subsystem 300 can receive a series of images captured by imaging device 220 of IVUS catheter 104 via image processing circuitry 116. Processor 302 can execute instructions 310 to receive information and/or data comprising indications of raw image frames 312.

Continuing to block 404 “pre-process and/or filter the series of images to generate a filtered series of images” the series of images received at block 402 can be pre-processed and/or filtered. For example, processor 302 can execute instructions 310 to filter the raw image frames 312 to generate filtered image frames 314. It is noted that block 404 is optional and, in some embodiments, logic flow 400 will proceed from block 402 to block 406. Continuing to block 406 “identify rotation between successive images of the filtered series of images” a rotation between successive images of the filtered series of images can be identified. For example, processor 302 can execute instructions 310 to identify a rotation between successive ones of filtered image frames 314. Alternatively, where block 404 is not executed, processor 302 can execute instructions 310 to identify a rotation between successive ones of raw image frames 312. In some examples, processor 302 can execute instructions 310 to determine a rotation threshold 318 based on a cross-correlation between filtered image frames 314 (or raw image frames 312 as may be the case).

FIG. 5A, FIG. 5B, and FIG. 5C illustrate successive images from a series of images captured by a rotational imaging device 220 of IVUS catheter 104 via image processing circuitry 116. For example, FIG. 5A illustrates a first image frame 502a; FIG. 5B illustrates a second image frame 502b; and FIG. 5C illustrates a third image frame 502c. The image frames 502a, 502b, and 502c can be successive images from a series of images (e.g., raw image frames 312, filtered image frames 314, etc.) As outlined above, at block 406, processor 302 can execute instructions 310 to identify a rotation between successive ones of a series of images frames. In some examples, processor 302 can execute instructions 310 to identify an artifact in the image frames 502a, 502b, and 502c and identify an angle of rotation between the artifacts. For example, image frames 502a, 502b, and 502c depict artifact 504, which is a shadow the guidewire over which the IVUS catheter 104 is inserted.

For example, processor 302 can execute instructions 310 to identify artifact 504 in first image frame 502a and second image frame 502b and to identify an angle of rotation between artifact 504 in these successive images. In such an example, processor 302 can execute instructions 310 to identify an angle of rotation between artifact 504 of first image frame 502a and second image frame 502b as 15 degrees. As a specific example, processor 302 can execute instructions 310 to determine that the artifact 504 in the first image frame 502a is at 150 degrees while the artifact 504 in the second image frame 502b is at 165 degrees. Further, processor 302 can execute instructions 310 to determine that the angle of rotation between the artifact 504 in these successive frames is 15 degrees. With some embodiments, processor 302 can execute instructions 310 to generate rotation between frames 316 comprising a matrix or listing of rotation angles.

Continuing with this example, processor 302 can execute instructions 310 to identify artifact 504 in the third image frame 502c and to identify an angle of rotation between artifact 504 in this image frame and the prior image frame 502b. Processor 302 can execute instructions 310 to determine that the artifact 504 in the third image frame 502c is at 178 degrees. Further, processor 302 can execute instructions 310 to determine that the angle of rotation between the artifact 504 in the third image frame 502c and the second image frame 502b is 13 degrees. With some embodiments, processor 302 can execute instructions 310 to update rotation between frames 316 comprising an indication of the angle of rotation between another pair of successive image frames. Accordingly, in this example, rotation between frames 316 would include an indication of 15 degrees (e.g., the rotation between artifact 504 in first and second image frames 502a and 502b) and 13 degrees (e.g., the rotation between artifact 504 in second and third image frames 502b and 502c).

It is noted that the above example provides first image frame 502a, second image frame 502b, and third image frame 502c. With some embodiments, the first, second, and third images frames 502a, 502b, and 502c can be successive in time, for example, captured at time t=1, t=2, and t=3. With some examples, the first, second, and third images frames 502a, 502b, and 502c can be successive in the sense they are similarly selected from a time series of image frames, for example, captured at time t=1, t=3, and t=5, captured at time t=1, t=4, t=7, or the like. That is, with some embodiments, the first, second, and third images frames 502a, 502b, and 502c need not be captured at adjacent time intervals but can instead be captured as proximal (e.g., within 1 frame, within 2 frames, within 3 frames, etc.) time intervals.

Returning to FIG. 4, logic flow 400 can continue from block 406 to decision block 408 “determine whether the rotation is greater than a threshold” a determination of whether the rotation is greater than a threshold can be determined. For example, processor 302 can execute instructions 310 to determine whether the rotation(s) indicated in rotation between frames 316 are greater than rotation threshold 318. In some examples, processor 302 can execute instructions 310 to determine whether the rotation(s) indicated in rotation between frames 316 are greater than or equal to the rotation threshold 318. In other examples, processor 302 can execute instructions 310 to determine whether the rotation(s) indicated in rotation between frames 316 are greater than rotation threshold 318. With some embodiments, rotation threshold 318 can be a range and processor 302 can execute instructions 310 to determine whether the rotation(s) indicated in rotation between frames 316 are greater than or equal to the lower bound of the range and less than or equal to the upper bound of the range.

In some examples, the rotation threshold 318 is 2.5 degrees. In some examples, the rotation threshold 318 is 5 degrees, 7.5 degrees, 10 degrees, or 12.5 degrees. In some examples, the rotation threshold 318 is between 2.5 and 30 degrees. In some examples, the rotation threshold 318 is between 5 and 25 degrees, 5 and 30 degrees, 5 and 45 degrees, 10 and 25 degrees, 10 and 30 degrees, or 10 and 45 degrees. With some examples, the rotation threshold 318 is set based upon the anatomy being imaged. For example, in the case of IVUS imaging systems (e.g., IVUS imaging system 100) there will be some natural rotation or movement of the artifact 504. As a specific example, during each cardiac cycle the heart will move, and this movement can appear as a rotation of the artifact. As such, the rotation threshold 318 can be set such that false positives are reduced, or rather, such that natural movement of the patient and/or anatomy is separated from rotational wind up of the image imaging core 204 and/or driveshaft 222.

With some examples, processor 302 can execute instructions 310 to complement the rotation between frames 316 with MDU state information 322. For example, multiple rotation thresholds 318 may be provided based on the speed and/or acceleration of the MDU 106. As a specific example, a smaller rotation threshold 318 may be provided where the acceleration is positive while a larger rotation threshold 318 may be provided where the acceleration is negative.

From decision block 408, logic flow 400 can continue to block 410 or return to block 402. For example, where at decision block 408 a determination is made that the rotation is greater than the threshold, logic flow 400 can continue to block 410 from decision block 408. At block 410 “generate a control signal comprising an indication to alert the user to the potential and/or actual twisting of the IVUS imaging device” a control signal comprising an indication to alert the user to the potential and/or actual twisting of the IVUS catheter 104 can be generated. For example, processor 302 can execute instructions 310 to generate a graphical alert to be displayed on a display where the graphical alert includes an indication of possible and/or actual twisting of the driveshaft 222 of the IVUS catheter 104. As another example, processor 302 can execute instructions 310 to generate an audible alert to be emitted by a speaker where the audible alert includes an indication of possible and/or actual twisting of the driveshaft 222 of the IVUS catheter 104.

From block 410, logic flow 400 can return to block 402. Alternatively, where at decision block 408 a determination is made that the rotation is not greater than the threshold, logic flow 400 can return to block 402 from decision block 408. In such a manner, logic flow 400 can be repeatedly or iteratively executed to identify the potential for and/or actual twist of an IVUS catheter 104 during a procedure as image frames are captured. In such an iterative example, at further instances of block 402, additional successive image frames (e.g., n+, or the like) can be received and the logic flow 400 can be implemented to determine whether there is a potential for twisting of kinking of the IVUS catheter 104 based on these additionally captured image frames.

Taking image frames 502a, 502b, and 502c as an example; logic flow 400 could be first executed and indications of image frames 502a and 502b could be received at block 402. On a second instance of executing block 402 of logic flow 400, image frame 502c could be received.

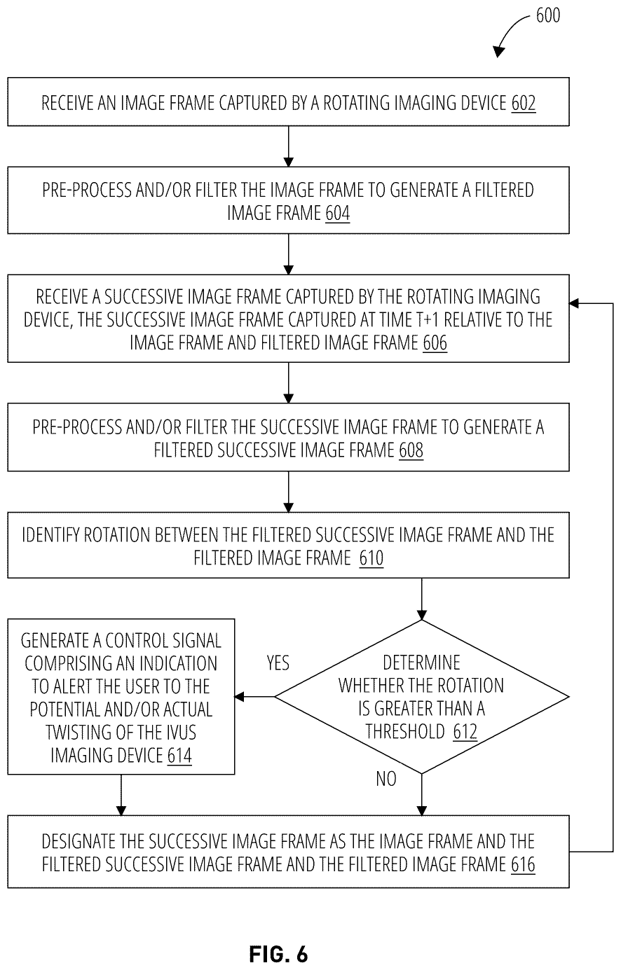

With some examples, rotation between successive image frames can be determined in a serial manner. For example, a frame can be received and compared with the previous in time frame. This is described in greater detail with reference to FIG. 6, which illustrates a logic flow 600 to identify potential and/or actual twisting of a rotational imaging driveshaft, according to some embodiments of the present disclosure. The logic flow 600 can be implemented by computer subsystem 300, which itself can be implemented by IVUS imaging system 100. Further, logic flow 600 will be described with reference to computer subsystem 300 for clarity of presentation. However, it is noted that logic flow 600 could also be implemented by an IVUS imaging system different than IVUS imaging system 100.

Logic flow 600 can begin at block 602. At block 602 “receive an image frame captured by a rotating imaging device” an image frame captured by a rotating imaging device can be received. For example, computer subsystem 300 can receive an image frame (e.g., one of raw image frames 312, or the like) captured by imaging device 220 of IVUS catheter 104 via image processing circuitry 116. Processor 302 can execute instructions 310 to receive information and/or data comprising indications of one image frame of raw image frames 312.

Continuing to block 604 “pre-process and/or filter the image frame to generate a filtered image frame” the image frame received at block 602 can be pre-processed and/or filtered to generate a filtered image frame. For example, processor 302 can execute instructions 310 to filter the image frame of raw image frames 312 to generate a filtered image frame (e.g., one of filtered image frames 314, or the like). It is noted that block 604 is optional and, in some embodiments, logic flow 600 will proceed from block 602 to block 606.

Continuing to block 606 “receive a successive image frame captured by the rotating imaging device, the successive image frame captured at time t+1 relative to the image frame and filtered image frame” a successive image frame captured by the rotating imaging device can be received. For example, computer subsystem 300 can receive a successive image frame (e.g., another one of raw image frames 312, or the like) captured by imaging device 220 of IVUS catheter 104 via image processing circuitry 116 where the successive image frame received at block 606 is captured at a time t+1 relative to the image frame and filtered image frame. Processor 302 can execute instructions 310 to receive information and/or data comprising indications of the other image frame (e.g., successive) of raw image frames 312.

Continuing to block 608 “pre-process and/or filter the successive image frame to generate a filtered successive image frame” the successive image frame received at block 606 can be pre-processed and/or filtered to generate a filtered successive image frame. For example, processor 302 can execute instructions 310 to filter the other image frame of raw image frames 312 to generate a filtered successive image frame (e.g., another one of filtered image frames 314, or the like). It is noted that block 608 is optional and, in some embodiments, logic flow 600 will proceed from block 608 to block 610.

Continuing to block 610 “identify rotation between the filtered successive image frame and the filtered image frame” a rotation between the filtered successive image frame and the filtered image frame can be identified. For example, processor 302 can execute instructions 310 to identify a rotation between the successive filtered image frames 314 (e.g., generated at block 608, or the like) and the filtered image frames 314 (e.g., generated at block 604, or the like). Alternatively, where blocks 604 and 608 are not executed, processor 302 can execute instructions 310 to identify a rotation between the successive raw image frames 312 (e.g., generated at block 606 and frame time t+1) and the raw image frames 312 (e.g., generated at block 602 and frame time t). In some examples, processor 302 can execute instructions 310 to determine a rotation threshold 318 based on a cross-correlation between the image frames.

Continuing to decision block 612 “determine whether the rotation is greater than a threshold” a determination of whether the rotation is greater than a threshold can be determined. For example, processor 302 can execute instructions 310 to determine whether the rotation identified at block 610 is greater than rotation threshold 318. In some examples, processor 302 can execute instructions 310 to determine whether the rotation (e.g., rotation between frames 316, or the like) is greater than or equal to the rotation threshold 318. In some examples, processor 302 can execute instructions 310 to determine a cumulative rotation (or average rotation) over several successive frames. For example, processor 302 can execute instructions 310 to determine whether the cumulative and/or average rotation between the prior 3 (4, 5, 6, etc.) successive frames exceeds the threshold.

With some examples, processor 302 can execute instructions 310 to complement the rotation between frames 316 with MDU state information 322. For example, multiple rotation thresholds 318 may be provided based on the speed and/or acceleration of the MDU 106. As a specific example, a smaller rotation threshold 318 may be provided where the acceleration is positive while a larger rotation threshold 318 may be provided where the acceleration is negative.

From decision block 612, logic flow 600 can continue to block 614 or block 616. For example, where at decision block 612 a determination is made that the rotation is greater than the threshold, logic flow 600 can continue to block 614 from decision block 612. At block 614 “generate a control signal comprising an indication to alert the user to the potential and/or actual twisting of the IVUS imaging device” a control signal comprising an indication to alert the user to the potential and/or actual twisting of the IVUS catheter 104 can be generated. For example, processor 302 can execute instructions 310 to generate a graphical alert to be displayed on a display where the graphical alert includes an indication of possible and/or actual twisting of the driveshaft 222 of the IVUS catheter 104. As another example, processor 302 can execute instructions 310 to generate an audible alert to be emitted by a speaker where the audible alert includes an indication of possible and/or actual twisting of the driveshaft 222 of the IVUS catheter 104.

Conversely, where at decision block 612 a determination is made that the rotation is not greater than the threshold, logic flow 600 can continue to block 616 from decision block 612. At block 616 “designate the successive image frame as the image frame and the filtered successive image frame and the filtered image frame” the successive image frame and the filtered successive image frame can be designated as the image frame and the filtered image frame. For example, processor 302 can execute instructions 310 to increment the time and t, as such, frames received at time t+1 will not be frames received at time t.

From block 616, logic flow 600 can return to block 606 where another successive image frame can be received and the logic flow 600 can continue to determine rotation between frames to identify potential twisting and/or buildup of rotational energy in the IVUS catheter 104.

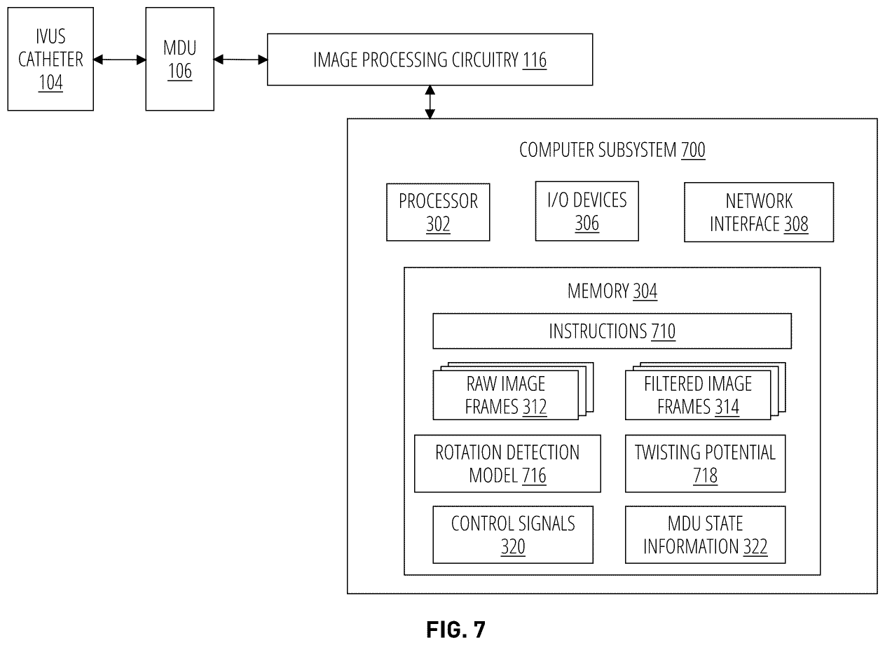

FIG. 7 illustrates an example of a computer subsystem 700, which can be implemented as part of the IVUS imaging system 100 of FIG. 1. For example, computer subsystem 700 could be implemented as computer subsystem 118 of IVUS imaging system 100 and configured to infer the potential for and/or actual twist of the driveshaft 222 using machine learning (ML). Computer subsystem 700 is depicted with some components of computer subsystem 300 and IVUS imaging system 100 for ease of description of brevity.

Like computer subsystem 300, computer subsystem 700 can be any of a variety of computing devices but will in general include processing circuitry and memory. With some examples, the processing circuitry (e.g., processor 302) can be and/or can include specialized processing circuitry for executing ML models. In computer subsystem 700, memory 304 can include instructions 710, raw image frames 312, filtered image frames 314, rotation detection model 716, twisting potential 718, control signals 320 and/or MDU state information 322.

During operation, processor 302 can execute instructions 710 to cause computer subsystem 700 to receive raw image frames 312 from image processing circuitry 116. Processor 302 can further execute instructions 710 to filter and/or pre-process raw image frames 312 to generate filtered image frames 314. Further, processor 302 can execute instructions 310 to generate twisting potential 718 from raw image frames 312 or filtered image frames 314 and rotation detection model 716. Said differently, processor 302 can execute instructions 310 to infer twisting potential 718 from rotation detection model 716 where raw image frames 312 or filtered image frames 314 are used as inputs to rotation detection model 716.

In some examples, rotation detection model 716 can be any of a variety of ML models. Rotation detection model 716 can be an image classification model, such as, a neural network (NN), a convolutional neural network (CNN), a random forest model, or the like. Generally, rotation detection model 716 is arranged to infer twisting potential 718 from raw image frames 312 or filtered image frames 314. For example, given a pair of successive image frames (e.g., first and second image frames 502a and 502b, second and third image frames 502b and 502c, or the like) rotation detection model 716 can infer a twisting potential 718. In some examples, twisting potential 718 is a binary twisting or no twisting detected. Rotation detection model 716 can be trained using any of a variety of training methodologies. Such as, for example, supervised or unsupervised learning. In a simple example, several sets of annotated image frames where the image frames are annotated to indicate whether they depict twisting buildup or not can be provided. Rotation detection model 716 could be trained using an optimization function and loss function to generate a trained rotation detection model 716 where it can infer from a series (or pair) of images whether twisting is building up or not.

With some examples, rotation detection model 716 can be configured to infer twisting potential 718 from filtered image frames 314 (or raw image frames 312 as may be the case) and MDU state information 322.

Processor 302 can execute instructions 710 to generate control signals 320 responsive to twisting potential 718 comprising an indication that twisting is likely, imminent, or already occurred.

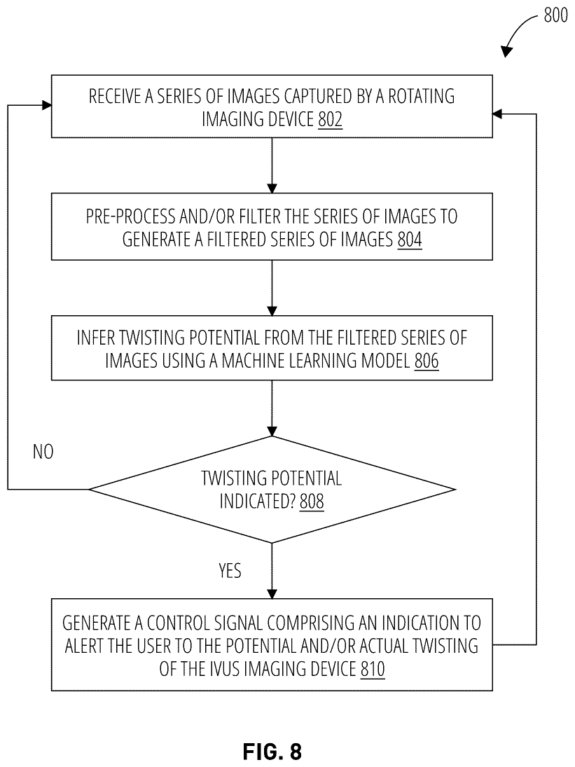

FIG. 8 illustrates a logic flow 800 to identify potential and/or actual twisting of a rotational imaging driveshaft, according to some embodiments of the present disclosure. The logic flow 800 can be implemented by computer subsystem 700, which itself can be implemented by IVUS imaging system 100. Further, logic flow 800 will be described with reference to computer subsystem 700 for clarity of presentation. However, it is noted that logic flow 800 could also be implemented by an IVUS imaging system different than IVUS imaging system 100.

Logic flow 800 can begin at block 802. At block 802 “receive a series of images captured by a rotating imaging device” a series of images captured by a rotating imaging device can be received. For example, computer subsystem 300 can receive a series of images captured by imaging device 220 of IVUS catheter 104 via image processing circuitry 116. Processor 302 can execute instructions 710 to receive information and/or data comprising indications of raw image frames 312.

Continuing to block 804 “pre-process and/or filter the series of images to generate a filtered series of images” the series of images received at block 802 can be pre-processed and/or filtered. For example, processor 302 can execute instructions 710 to filter the raw image frames 312 to generate filtered image frames 314. It is noted that block 804 is optional and, in some embodiments, logic flow 800 will proceed from block 802 to block 806.

Continuing to block 806 “infer twisting potential from the filtered series of images using a machine learning model” a twisting potential can be inferred from the filtered series of images (or the raw images as may be the case) using an ML model. For example, processor 302 can execute instructions 710 to generate (or infer) twisting potential 718 from filtered image frames 314 using rotation detection model 716. Alternatively, where block 804 is not executed, processor 302 can execute instructions 710 to generate (or infer) twisting potential 718 from raw image frames 312 using rotation detection model 716. As another example, processor 302 can execute instructions 710 to generate (or infer) twisting potential 718 from filtered image frames 314 and MDU state information 322 using rotation detection model 716.

Continuing to decision block 808 “twisting potential indicated?” a determination of whether twisting potential is indicated by twisting potential 718 can be made. For example, processor 302 can execute instructions 710 to determine whether the twisting potential 718 has a confidence level above a threshold. From decision block 808, logic flow 800 can continue to block 810 or return to block 802. For example, where at decision block 808 a determination is made that the twisting potential is indicated, logic flow 800 can continue to block 810 from decision block 808. At block 810 “generate a control signal comprising an indication to alert the user to the potential and/or actual twisting of the IVUS imaging device” a control signal comprising an indication to alert the user to the potential and/or actual twisting of the IVUS catheter 104 can be generated. For example, processor 302 can execute instructions 710 to generate a graphical alert to be displayed on a display where the graphical alert includes an indication of possible and/or actual twisting of the driveshaft 222 of the IVUS catheter 104. As another example, processor 302 can execute instructions 710 to generate an audible alert to be emitted by a speaker where the audible alert includes an indication of possible and/or actual twisting of the driveshaft 222 of the IVUS catheter 104.

From block 810, logic flow 800 can return to block 802. Alternatively, where at decision block 808 a determination is made that the twisting potential is not indicated, logic flow 800 can return to block 802 from decision block 808. In such a manner, logic flow 800 can be repeatedly or iteratively executed to identify the potential for and/or actual twist of an IVUS catheter 104 during a procedure as image frames are captured. In such an iterative example, at further instances of block 802, additional successive image frames (e.g., n+, or the like) can be received and the logic flow 800 can be implemented to determine whether there is a potential for twisting of kinking of the IVUS catheter 104 based on these additionally captured image frames.

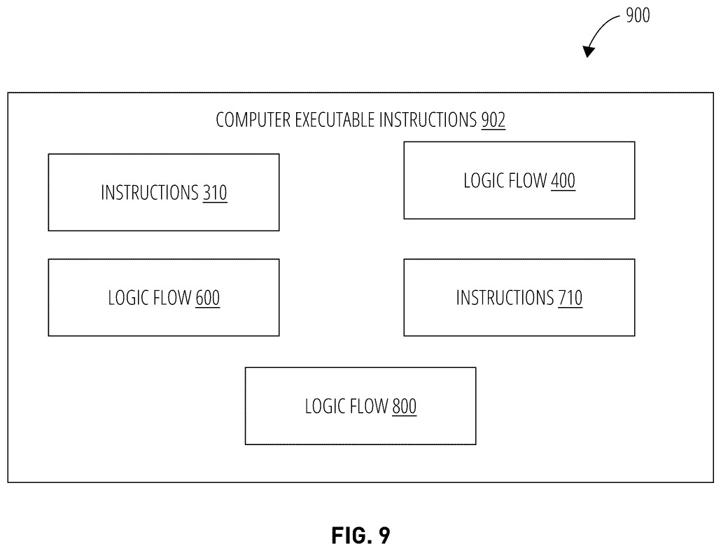

FIG. 9 illustrates computer-readable storage medium 900. Computer-readable storage medium 900 may comprise any non-transitory computer-readable storage medium or machine-readable storage medium, such as an optical, magnetic or semiconductor storage medium. In various embodiments, computer-readable storage medium 900 may comprise an article of manufacture. In some embodiments, computer-readable storage medium 900 may store computer executable instructions 902 with which circuitry (e.g., processor 302, or the like) can execute. For example, computer executable instructions 902 can include instructions to implement operations described with respect to instructions 310, logic flow 400, logic flow 600 instructions 710, and/or logic flow 800. Examples of computer-readable storage medium 900 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 902 may include any suitable type of code, such as source code, compiled code, interpreted code, executable code, static code, dynamic code, object-oriented code, visual code, and the like.

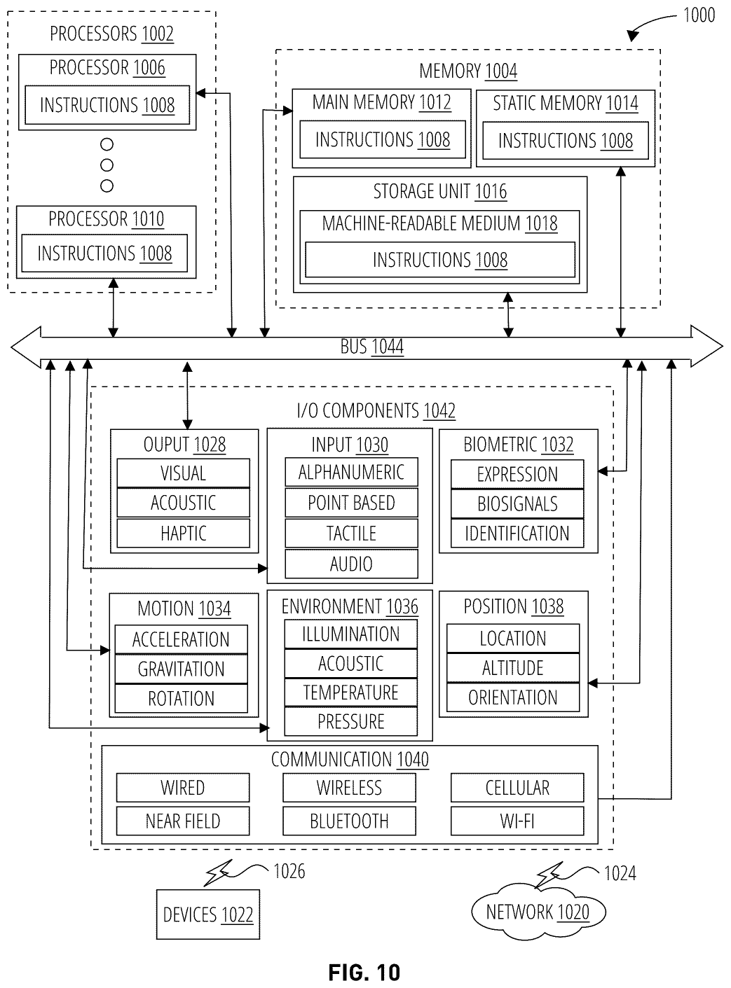

FIG. 10 illustrates a diagrammatic representation of a machine 1000 in the form of a computer system within which a set of instructions may be executed for causing the machine to perform any one or more of the methodologies discussed herein. More specifically, FIG. 10 shows a diagrammatic representation of the machine 1000 in the example form of a computer system, within which instructions 1008 (e.g., software, a program, an application, an applet, an app, or other executable code) for causing the machine 1000 to perform any one or more of the methodologies discussed herein may be executed. For example, the instructions 1008 may cause the machine 1000 to execute logic flow 400 of FIG. 4, logic flow 800 of FIG. 8, or the like. More generally, the instructions 1008 may cause the machine 1000 to identify potential and/or actual twisting of the driveshaft 222 of IVUS catheter 104 and provide an alert to the user. In such a manner, the user can be provided an opportunity to recover the situation (e.g., by backing out the catheter, unwinding the core, or the like).

The instructions 1008 transform the general, non-programmed machine 1000 into a particular machine 1000 programmed to carry out the described and illustrated functions in a specific manner. In alternative embodiments, the machine 1000 operates as a standalone device or may be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1008, sequentially or otherwise, that specify actions to be taken by the machine 1000. Further, while only a single machine 1000 is illustrated, the term “machine” shall also be taken to include a collection of machines 200 that individually or jointly execute the instructions 1008 to perform any one or more of the methodologies discussed herein.

The machine 1000 may include processors 1002, memory 1004, and I/O components 1042, which may be configured to communicate with each other such as via a bus 1044. In an example embodiment, the processors 1002 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1006 and a processor 1010 that may execute the instructions 1008. The term “processor” is intended to include multi-core processors that may comprise two or more independent processors (sometimes referred to as “cores”) that may execute instructions contemporaneously. Although FIG. 10 shows multiple processors 1002, the machine 1000 may include a single processor with a single core, a single processor with multiple cores (e.g., a multi-core processor), multiple processors with a single core, multiple processors with multiples cores, or any combination thereof.

The memory 1004 may include a main memory 1012, a static memory 1014, and a storage unit 1016, both accessible to the processors 1002 such as via the bus 1044. The main memory 1004, the static memory 1014, and storage unit 1016 store the instructions 1008 embodying any one or more of the methodologies or functions described herein. The instructions 1008 may also reside, completely or partially, within the main memory 1012, within the static memory 1014, within machine-readable medium 1018 within the storage unit 1016, within at least one of the processors 1002 (e.g., within the processor's cache memory), or any suitable combination thereof, during execution thereof by the machine 1000.

The I/O components 1042 may include a wide variety of components to receive input, provide output, produce output, transmit information, exchange information, capture measurements, and so on. The specific I/O components 1042 that are included in a particular machine will depend on the type of machine. For example, portable machines such as mobile phones will likely include a touch input device or other such input mechanisms, while a headless server machine will likely not include such a touch input device. It will be appreciated that the I/O components 1042 may include many other components that are not shown in FIG. 10. The I/O components 1042 are grouped according to functionality merely for simplifying the following discussion and the grouping is in no way limiting. In various example embodiments, the I/O components 1042 may include output components 1028 and input components 1030. The output components 1028 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1030 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1042 may include biometric components 1032, motion components 1034, environmental components 1036, or position components 1038, among a wide array of other components. For example, the biometric components 1032 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1034 may include acceleration sensor components (e.g., accelerometer), gravitation sensor components, rotation sensor components (e.g., gyroscope), and so forth. The environmental components 1036 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1038 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1042 may include communication components 1040 operable to couple the machine 1000 to a network 1020 or devices 1022 via a coupling 1024 and a coupling 1026, respectively. For example, the communication components 1040 may include a network interface component or another suitable device to interface with the network 1020. In further examples, the communication components 1040 may include wired communication components, wireless communication components, cellular communication components, Near Field Communication (NFC) components, Bluetooth® components, Wi-Fi® components, and other communication components to provide communication via other modalities. The devices 1022 may be another machine or any of a wide variety of peripheral devices (e.g., a peripheral device coupled via a USB).

Moreover, the communication components 1040 may detect identifiers or include components operable to detect identifiers. For example, the communication components 1040 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes, and other optical codes), or acoustic detection components (e.g., microphones to identify tagged audio signals). In addition, a variety of information may be derived via the communication components 1040.

The various memories (i.e., memory 1004, main memory 1012, static memory 1014, and/or memory of the processors 1002) and/or storage unit 1016 may store one or more sets of instructions and data structures (e.g., software) embodying or utilized by any one or more of the methodologies or functions described herein. These instructions (e.g., the instructions 1008), when executed by processors 1002, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1020 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1020 or a portion of the network 1020 may include a wireless or cellular network, and the coupling 1024 may be a Code Division Multiple Access (CDMA) connection, a Global System for Mobile communications (GSM) connection, or another type of cellular or wireless coupling. In this example, the coupling 1024 may implement any of a variety of types of data transfer technology.

The instructions 1008 may be transmitted or received over the network 1020 using a transmission medium via a network interface device (e.g., a network interface component included in the communication components 1040) and utilizing any one of several well-known transfer protocols (e.g., hypertext transfer protocol (HTTP)). Similarly, the instructions 1008 may be transmitted or received using a transmission medium via the coupling 1026 (e.g., a peer-to-peer coupling) to the devices 1022. The terms “transmission medium” and “signal medium” mean the same thing and may be used interchangeably in this disclosure. The terms “transmission medium” and “signal medium” shall be taken to include any intangible medium that can store, encoding, or carrying the instructions 1008 for execution by the machine 1000, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A computer-implemented method to provide an alert of potential twisting of a rotational imaging device, comprising:
receiving a series of image frames captured by a rotational imaging device, where the series of image frames comprises at least a first image frame and a second image frame successive to the first image frame;
identifying an angle of rotation between the first image frame and the second image frame; and
generating, responsive to the determined angle of rotation, a control signal comprising an indication of a potential twisting of the rotational imaging device.

2. The computer-implemented method of claim 1, comprising cross-correlating the first image frame and the second image frame to identify the angle of rotation.

3. The computer-implemented method of claim 16, comprising filtering the series of image frames to generate a filtered series of image frames, wherein identifying the angle of rotation between the first image frame and the second image frame comprising identifying the angle of rotation from the filtered series of image frames.

4. The computer-implemented method of claim 1, wherein the control signal comprises an indication of a graphical alert to be displayed on a display.

5. The computer-implemented method of claim 1, comprising:
determining whether the determined angle of rotation is greater than or equal to a rotation threshold; and
generating the control signal based on a determination that the determined angle of rotation is greater than or equal to the rotation threshold.

6. The computer-implemented method of claim 1, wherein the angle of rotation is a first angle of rotation, wherein the series of image frames comprises at least a third image frame successive to the second image frame, and wherein the method comprises:
identifying a second angle of rotation between the second image frame and the third image frame; and
generating the control signal responsive to the first and the second angles of rotation.

7. The computer-implemented method of claim 1, wherein the rotational imaging device is an intravascular ultrasound (IVUS) catheter comprising a distal imaging core coupled to a proximal motor drive unit connector via a driveshaft and wherein the potential twisting of the rotational imaging device corresponds to a potential winding up of the driveshaft.

8. An intravascular ultrasound (IVUS) imaging system configured to be coupled to a motor drive unit and an IVUS catheter, the IVUS imaging system comprising:
processing circuitry and a memory comprising instructions, which when executed cause the IVUS imaging system to:
receive, from the IVUS catheter, a series of image frames captured by the IVUS catheter, where the series of image frames comprises at least a first image frame and a second image frame successive to the first image frame;
identify an angle of rotation between the first image frame and the second image frame;
generate, responsive to the determined angle of rotation, a graphical indication of a potential twisting of the IVUS catheter; and
display on a display coupled to the IVUS imaging system the graphical indication.

9. The IVUS imaging system of claim 8, the instructions when executed by the processing circuitry further cause the IVUS imaging system to cross-correlate the first image frame and the second image frame to identify the angle of rotation.

10. The IVUS imaging system of claim 8, the instructions when executed by the processing circuitry further cause the IVUS imaging system to filter the series of image frames to generate a filtered series of image frames, wherein the angle of rotation between the first image frame and the second image frame is identified from the filtered series of image frames.

11. The IVUS imaging system of claim 8, the instructions when executed by the processing circuitry further cause the IVUS imaging system to:
determine whether the determined angle of rotation is greater than or equal to a rotation threshold; and
generate the control signal based on a determination that the determined angle of rotation is greater than or equal to the rotation threshold.

12. The IVUS imaging system of claim 8, wherein the angle of rotation is a first angle of rotation, wherein the series of image frames comprises at least a third image frame successive to the second image frame, and wherein the instructions when executed by the processing circuitry further cause the IVUS imaging system to:
identify a second angle of rotation between the second image frame and the third image frame; and
generate the control signal responsive to the first and the second angles of rotation.

13. The IVUS imaging system of claim 8, wherein the IVUS catheter comprises a distal imaging core coupled to a proximal motor drive unit connector via a driveshaft and wherein the potential twisting of the IVUS catheter corresponds to a potential winding up of the driveshaft.

14. The IVUS imaging system of claim 8, comprising the IVUS catheter.

15. The IVUS imaging system of claim 8, comprising the motor drive unit and the IVUS catheter.

16. A non-transitory computer-readable storage device, comprising instructions that when executed by a processor of an intravascular ultrasound (IVUS) imaging system cause the IVUS imaging system to:
receive, from an IVUS catheter coupled to a motor drive unit, a series of image frames captured by the IVUS catheter, where the series of image frames comprises at least a first image frame and a second image frame successive to the first image frame;
identify an angle of rotation between the first image frame and the second image frame;
generate, responsive to the determined angle of rotation, a graphical indication of a potential twisting of the IVUS catheter; and
display on a display coupled to the IVUS imaging system the graphical indication.

17. The non-transitory computer-readable storage device of claim 16, wherein the instructions when executed by the processor further cause the IVUS imaging system to cross-correlate the first image frame and the second image frame to identify the angle of rotation.

18. The non-transitory computer-readable storage device of claim 16, wherein the instructions when executed by the processor further cause the IVUS imaging system to filter the series of image frames to generate a filtered series of image frames, wherein the angle of rotation between the first image frame and the second image frame is identified from the filtered series of image frames.

19. The non-transitory computer-readable storage device of claim 16, wherein the instructions when executed by the processing circuitry further cause the IVUS imaging system to:
determine whether the determined angle of rotation is greater than or equal to a rotation threshold; and
generate the control signal based on a determination that the determined angle of rotation is greater than or equal to the rotation threshold.

20. The non-transitory computer-readable storage device of claim 16, wherein the IVUS catheter comprises a distal imaging core coupled to a proximal motor drive unit connector via a driveshaft and wherein the potential twisting of the IVUS catheter corresponds to a potential winding up of the driveshaft.
